(19) United States
(12) Patent Application Publication
(10) Pub. No.: US 2010/0158994 A1
(43) Pub. Date: Jun. 24, 2010

(54) Nanoparticles for Delivery of Therapeutic Agents Using Ultrasound and Associated Methods

(76) Inventor: Kenneth L. Watkin, Champaign, IL (US)

Correspondence Address: ECKERT SEAMANS CHERIN & MELLOTT, 600 GRANT STREET, 44TH FLOOR, PITTSBURGH, PA, 15219, US

(21) Appl. No.: 12/694,891

(22) Filed: Jan. 27, 2010

Related U.S. Application Data
(62) Division of application No. 11/271,641, filed on Nov. 10, 2005.
(60) Provisional application No. 60/635,838, filed on Dec. 14, 2004.

(51) Int. Cl. A61K 9/127; A61K 39/44; A61K 35/76; A61K 35/12; A61K 38/02; A61K 31/7088; A61K 31/70

(52) U.S. Cl. 424/450; 424/178.1; 424/93.2; 424/93.7; 514/2; 514/44 R; 514/23; 514/44 A; 977/773; 977/907

(57) ABSTRACT

The present invention relates to lipid based nanoparticles or liposomes that are sensitive to ultrasonic energy, compositions containing these particles, methods for delivering one or more active agents using the particles, and methods for preparing the particles. The nanoparticles and liposomes encapsulate active agents such as chemotoxins, genes, virus vectors, proteins, peptides, antisense oligonucleotides, carbohydrates, and stem cells. The particles contain an aqueous core, at least one active agent located within the aqueous core, and a lipid bilayer or membrane that encapsulates the active agent within the aqueous core. The lipid bilayer may comprise a primary phospholipid and a lysolipid that preferably have different acyl chain lengths, making the lipid bilayer sensitive to ultrasound. Ultrasound may be used to track the particles as they move throughout the body. When the ultrasonic energy reaches a certain pressure, the lipid bilayer will break apart, releasing the active agent.

### FIELD OF THE INVENTION

The present invention relates to lipid based nanoparticles or liposomes that are sensitive to ultrasonic energy, and more particularly relates to the use of these nanoparticles or liposomes to carry and release active agents such as chemotoxins, genes, virus vectors, proteins, peptides, antisense oligonucleotides, carbohydrates, and stem cells.

### BACKGROUND INFORMATION

Nanoparticles and liposomes serve as ideal carriers for the delivery of payloads such as chemotoxins, genes, virus vectors, proteins, peptides, antisense oligonucleotides, carbohydrates, stem cells, and/or other agents for the treatment of disease. In general, a nanoparticle or liposome is a spherical vesicle with a membrane composed of a lipid bilayer that encapsulates an active agent, e.g., a therapeutic agent such as a drug or genetic material. When the lipid bilayer dissolves or breaks apart, the active agent is released into the body. Nanoparticles have a width of less than about 500 nm, and liposomes have a width of greater than about 500 nm but less than about 30 μm. Because of their small size, nanoparticles and liposomes can be injected directly into muscles, joints, or the peritoneum, or orally administered using a capsule that releases the particles at a predetermined pH or temperature level. Ligands may be attached to the surfaces of the particles to assist in reaching targeted treatment sites.

There are two types of drug delivery systems—passive and active. Passive delivery systems rely on the passive assimilation of a payload in the vicinity of a treatment site; the drug is not actively directed to the site. Active delivery systems rely on the manipulation of a carrier vehicle, such as a nanoparticle, to direct the payload to the treatment site. The payload may be released using changes in pH or temperature. If a nanoparticle is pH-sensitive, the payload is released when the environment reaches a transitional pH. If a nanoparticle is temperature-sensitive, the payload is released when the environment reaches a transitional temperature. However, one of the drawbacks to using pH and/or temperature as the release mechanism is the inability to visualize the targeted region while at the same time controlling the release of the payload.

Another problem with current microspheres, liposomes, and nanoparticles is that they are formed using perfluorocarbon gas; the active agents are bound on the surface of the particle or within the layers forming the membrane or shell of the particle. The amount of active agent that can be delivered is limited to the amount that is adhered to the surface of the particle or bound into the shell.

Thus, there exists a need for a carrier vehicle that can encapsulate active agents within the aqueous core of a nanoparticle or liposome and allow for tracking, targeting, and release of the agent using imaging technology such as ultrasound. Such a carrier vehicle would provide advanced control for the delivery of active agents, as well as other patient and industrial advantages. For example, the carrier vehicle would reduce the circulating levels and amounts of active agents that are required for treatment by providing particles capable of encapsulating greater amounts of the active agents. It would also reduce the time required for treatment, lower the costs associated with pharmaceutical production, and facilitate the encapsulation of exotic materials such as plant extracts.

### SUMMARY OF THE INVENTION

The present invention provides lipid based nanoparticles or liposomes that are sensitive to ultrasonic energy, compositions containing these particles, methods for delivering the particles to various treatment sites in the body, and methods for preparing the particles. The particles encapsulate active agents such as chemotoxins, genes, virus vectors, proteins, peptides, antisense oligonucleotides, carbohydrates, and stem cells, carry the active agents throughout the body, and release the active agents when subjected to pulses of ultrasonic energy. The active agents are encapsulated within the aqueous core of the particle by a lipid bilayer or membrane comprising a primary phospholipid and a lysolipid. The primary phospholipid and the lysolipid preferably have different acyl chain lengths, which makes the lipid bilayer sensitive to ultrasound. Ultrasound may be used to track the particles as they move throughout the body. When the ultrasonic energy reaches a certain pressure, the lipid bilayer will break apart, releasing the active agent into the body.

An object of the present invention is to provide a composition for delivering at least one active agent, the composition comprising at least one particle having an aqueous core and a lipid bilayer, wherein the lipid bilayer encapsulates the at least one active agent within the aqueous core.

Another object of the present invention is to provide a method for delivering at least one active agent, the method comprising administering at least one particle to a patient, wherein the at least one particle includes an aqueous core and a lipid bilayer that encapsulates the at least one active agent within the aqueous core; tracking the movement of the particle; and releasing the active agent from the particle using ultrasound.

A further object of the present invention is to provide a method for preparing at least one particle having an aqueous core and a lipid bilayer, wherein the lipid bilayer encapsulates at least one active agent within the aqueous core, the method comprising: combining a primary phospholipid and a lysolipid to form the lipid bilayer; producing a film of the lipid bilayer; introducing the at least one active agent to the film of lipid bilayer; applying sonication to the film of lipid bilayer and the active agent to form at least one particle; and removing active agent that is not encapsulated within a particle following sonication.

Another object of the present invention is to reduce the circulating levels and amounts of active agents that are required for treatment by providing particles capable of encapsulating greater amounts of the active agents.

Another object of the present invention is to reduce the time required for treatment by providing a method for releasing encapsulated agents using ultrasound.

A further object of the present invention is to lower pharmaceutical production costs by providing particles capable of encapsulating greater amounts of the active agents.

Another object of the present invention is to provide particles that allow for the encapsulation of exotic materials.

These and other aspects of the present invention will become more readily apparent from the following detailed description and appended claims.

### FIGURES

FIGS. 1a-b depict a molecule of DPPC and its molecular structure, respectively.

FIGS. 2a-b depict a molecule of DSPC and its molecular structure, respectively.

FIGS. 3a-b depict a molecule of MMPC and its molecular structure, respectively.

FIGS. 4a-b depict a molecule of MPPC and its molecular structure, respectively.

FIG. 5 provides a chart showing percent release of an encapsulated carboxyfluorescein for three different types of nanoparticles.

FIG. 6 provides a chart showing percent release of an encapsulated carboxyfluorescein for three different types of nanoparticles.

### DETAILED DESCRIPTION OF THE PREFERRED EMBODIMENTS

As employed herein, the term “patient” refers to members of the animal kingdom including humans.

As employed herein, the term “particle” shall refer to nanoparticles, which have a width of less than about 500 nm, as well as liposomes, which have a width ranging from about 500 nm to 30 μm.

As employed herein, the term “active agent” shall refer to an encapsulated agent that assists in treating or preventing a condition, including but not limited to chemotoxins, genes, virus vectors, proteins, peptides, antisense oligonucleotides, carbohydrates, stem cells, and other suitable agents for the treatment and/or prevention of conditions.

As employed herein, the term “acyl chain” shall refer to the hydrocarbon chain or tail attached to a molecule.

As employed herein, the term “acyl chain length” or “hydrocarbon chain length” shall refer to the number of carbon atoms that comprise an acyl chain.

The present invention provides lipid based nanoparticles or liposomes that are sensitive to ultrasonic energy, compositions containing the particles, methods for delivering active agents using the particles, and methods for preparing the particles. As used herein, the term “particle” refers to nanoparticles, which have a width of less than about 500 nm, as well as liposomes, which have a width ranging from about 500 nm to 30 μm. The particles may be used to treat or prevent a variety of conditions, including but not limited to cancer, cardiovascular disease, atherosclerosis, vulnerable plaque, arthritis, and gliomas. The particles will encapsulate one or more active agents, such as chemotoxins, genes, virus vectors, proteins, peptides, antisense oligonucleotides, carbohydrates, and stem cells, that are used for the treatment or prevention of various medical conditions. The active agents are encapsulated within the aqueous core of the particles and are surrounded by a lipid bilayer or membrane that may comprise a primary phospholipid and a lysolipid. The primary phospholipid and the lysolipid preferably have different acyl chain lengths, which makes the lipid bilayer sensitive to ultrasound. Ultrasound may be used to track the particles as they move throughout the body. When the ultrasonic energy reaches a certain pressure, the lipid bilayer will break apart, releasing the active agents into the body.

A nanoparticle or liposome is a spherical vesicle with an aqueous core and a membrane composed of a lipid bilayer that encapsulates one or more active agents within the aqueous core. A lipid bilayer is a membrane or zone of membrane composed of two opposing layers of lipid molecules. The molecules are arranged so that their hydrocarbon tails face one another to form an oily bilayer. The hydrocarbon tails are also referred to as “hydrocarbon chains” or “acyl chains.” The molecules have electrically charged or polar heads that face the aqueous core on one side of the membrane. According to the present invention, the molecules of the lipid bilayer may comprise a primary phospholipid and a lysolipid. The primary phospholipid may have a hydrocarbon chain length that differs from the hydrocarbon chain length of the lysolipid. The primary phospholipid may have a chain length ranging from about 6 to 20 carbon atoms, with a preferred chain length of about 18 carbon atoms. The lysolipid may have a chain length ranging from about 6 to 24 carbon atoms, with a preferred chain length of about 14 carbon atoms. In a preferred embodiment, the difference in chain length between the primary phospholipid and lysolipid is about 4 carbon atoms.

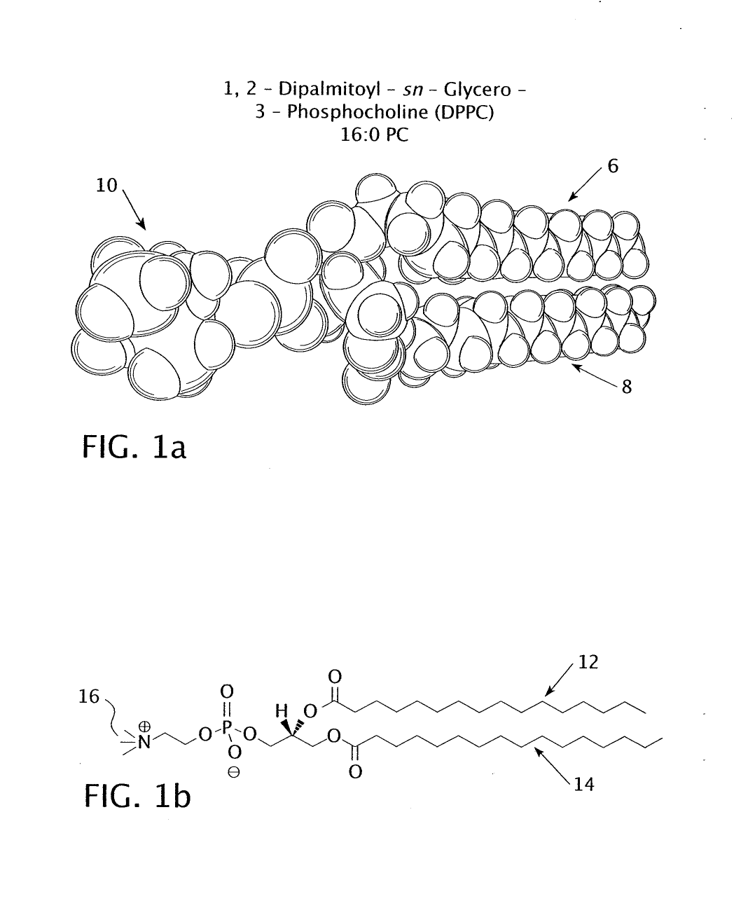

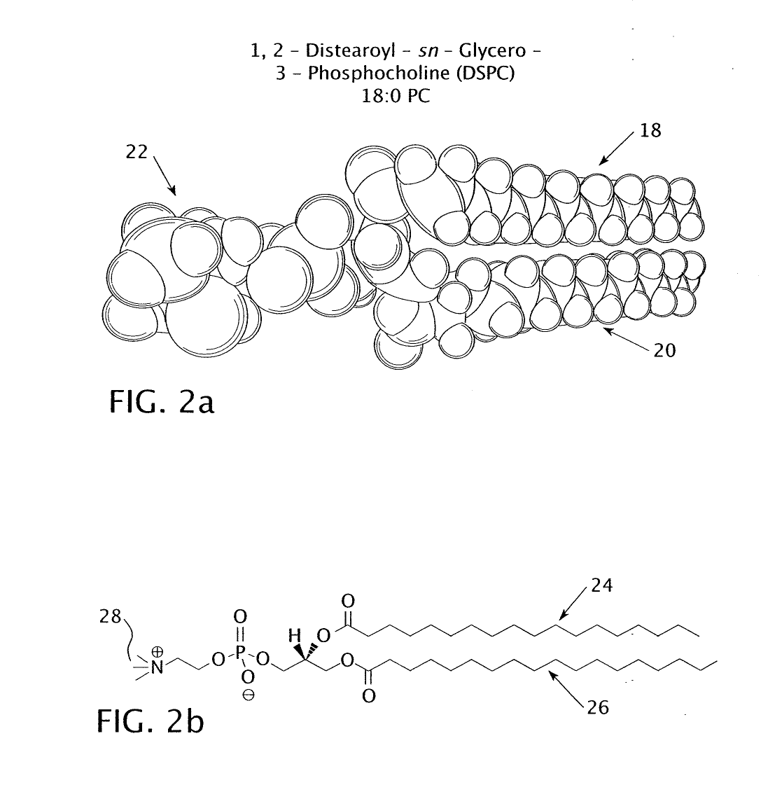

The primary phospholipid may comprise a di-chain phospholipid, for example, 1,2-dipalmitoyl-sn-glycero-3-phosphocholine (DPPC), or in a preferred embodiment, 1,2-di-distearoyl-sn-glycero-3-phosphocholine (DSPC). The primary phospholipid may also comprise a tri-chain phospholipid, or any other suitable phospholipid with a plurality of acyl chains. FIG. 1 a depicts a molecule of DPPC, which has two hydrocarbon chains 6, 8 that are attached to an electrically charged head 10. FIG. 1 b depicts the molecular structure for DPPC, showing the two hydrocarbon chains 12, 14 and electrically charged head 16. FIG. 2a depicts a molecule of DSPC, which has two hydrocarbon chains 18, 20 attached to an electrically charged head 22. FIG. 2b depicts the molecular structure for DSPC, showing the two hydrocarbon chains 24, 26 and electrically charged head 28.

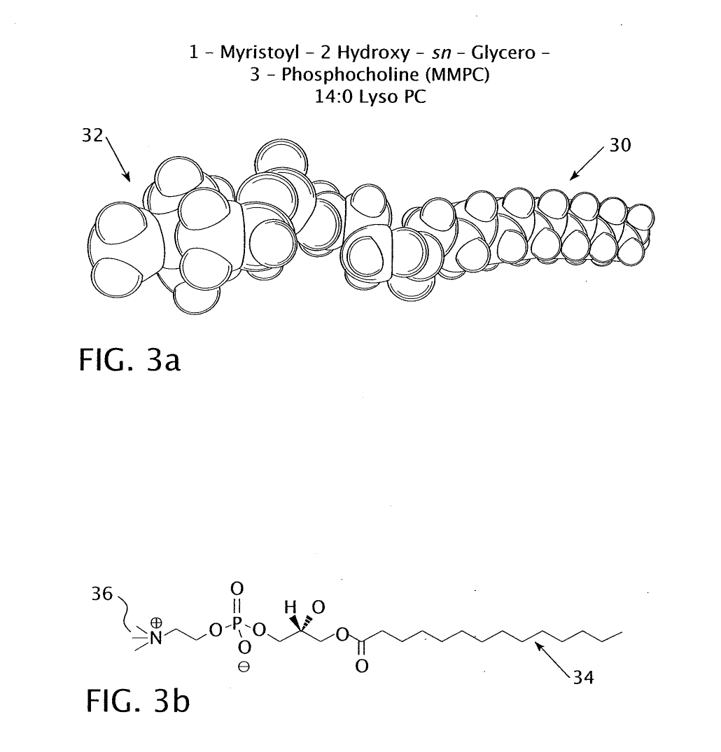

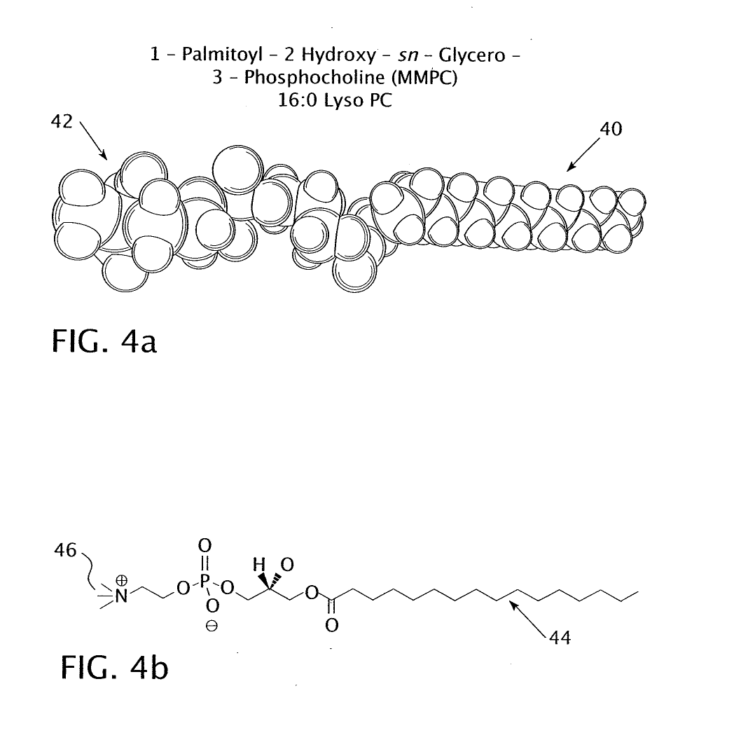

The lysolipid may comprise a molecule with a single acyl chain. The molecule may be a derivative of a phosphatic acid that lacks one of its fatty acid chains due to hydrolytic removal. The lysolipid may comprise a C6-C20 monoacyl lysolipid, and preferably comprises a surface active agent such as 1-myristoy1-2-hydroxy-sn-glycero-3-phosphocholine (MMPC) or 1-palmitoy1-2-hydroxy-sn-glycero-3-phosphocholine (MPPC). Other examples of lysolipids include 1-oleoy1-2-hydroxy-sn-glycero-3-phosphocholine (MOPC), 1-lauroy1-2-hydroxy-sn-glycero-3-phosphocholine (MLPC), and 1-stearoy1-2-hydroxy-sn-glycero-3-phosphocholine (MSPC), or any other suitable mono-chain lysolipid. FIG. 3a depicts a molecule of MMPC, which has a single hydrocarbon chain 30 attached to an electrically charged head 32. FIG. 3b depicts the molecular structure for MMPC, showing the single hydrocarbon chain 34 and electrically charged head 36. FIG. 4a depicts a molecule of MPPC, which has a single hydrocarbon chain 40 attached to an electrically charged head 42. FIG. 4b depicts the molecular structure for MMPC, showing the single hydrocarbon chain 44 and electrically charged head 46.

The primary phospholipid and the lysolipid organize to form a lipid bilayer. The primary phospholipid may comprise from about 80% to 95% of the bilayer, and preferably comprises about 90% of the bilayer. The molar ratio of the primary phospholipid to the lysolipid may range from about 80:20 to about 95:5, and is preferably about 90:10. The bilayer may also contain other substances such as cholesterol, a surface coating of polyethylene glycol, or another polymer such as dextran. In a preferred embodiment, the bilayer may comprise a phospholipid with a transition temperature greater than about 50 degrees Celsius, lysolipid, cholesterol, and pegylated. Lipids such as phosphocholine (PC) have a transition phase or temperature (T_{c}) at which they become gel like and unstable. To ensure stability within the body, the transition temperature of the PC should be greater than normal human body temperature, which is about 37 degrees Celsius, and the average temperature of a feverish human body, which is about 39 degrees Celsius. The transition temperature of the PC should also be greater than about 45 degrees Celsius to ensure stability in high temperature climates. Thus, the transition temperature of the PC should be greater than about 50 degrees Celsius, but less than about 55.1 degrees, which is the transition temperature for DSPC.

The chain length of the primary phospholipid differs from the chain length of the lysolipid, making the bilayer susceptible to increases in ultrasonic pressure. The primary phospholipid may have a chain length of about 6 to 20 carbon atoms, with a preferred chain length of 18 carbon atoms. The lysolipid may have a chain length of about 6 to 24 carbon atoms, with a preferred chain length of 14 carbon atoms. In a preferred embodiment, the primary phospholipid may comprise DSPC with a chain length of 18, and the lysolipid may comprise MMPC with a chain length of 14; the molar ratio of DSPC to MMPC is about 90:10. In another embodiment, the primary phospholipid may comprise DSPC with a chain length of 16, and the lysolipid may comprise MMPC with a chain length of 14. The molar ratio of DSPC to MMPC may range from about 95:5 to 80:20.

The difference in chain length between the primary phospholipid and the lysolipid makes the bilayer sensitive to ultrasonic energy. Ultrasound waves are reflected by the bilayer and are capable of detecting the difference in chain length. At a certain pressure, the ultrasound waves will break the bilayer, releasing the active agents contained in the core. The ultrasound may also break the bilayer when the chain lengths of the primary phospholipid and lysolipid are identical. However, the amount of active agents released is significantly reduced when the chain lengths are the same.

The particles may be administered to the patient using injection, oral administration, aerosols, or any other suitable method. In a preferred embodiment, the particles are injected into the muscles, joints, or peritoneum. The size of the particles will depend on the proposed use. In one embodiment, the average size of each particle may range from about 30 nm to 5000 nm, with a preferred range of about 100-200 nm. Once the particles are administered, the clinician may use commercially available ultrasound equipment, e.g., a diagnostic medical ultrasound machine and probe, to track the movement of the particles throughout the patient's body. For tracking purposes, the ultrasound is operated at “tracking pressure” that typically ranges from about 1½ to 2½ MPa, but may reach up to 4 MPa. To assist in directing the particle to a specific treatment site in the patient, a ligand may be attached to the surface of the particles. In one embodiment, the ligand comprises monoclonal antibodies that attach to their antigens. To release the active agents at a particular site, the ultrasonic pressure may be increased to a “release pressure” that is higher than the “tracking pressure.” The release pressure is typically around 3 MPa, although it may range from about 1½ MPa to 5 MPa. The release pressure is typically maintained for a duration of approximately 100-900 milliseconds before the lipid bilayer breaks apart, with a preferred duration of 500 milliseconds. The frequency of the ultrasound waves for both tracking and release purposes is typically maintained at around 2-20 MHz, with a preferred frequency of 7.5 MHz.

The particles may be prepared by combining a primary phospholipid and a lysolipid to form the lipid bilayer; producing a film of the lipid bilayer; introducing the at least one active agent to the film of lipid bilayer; applying sonication to the film of lipid bilayer and the active agent to form at least one particle; and removing active agent that is not encapsulated within a particle following sonication. In a preferred embodiment, the primary phospholipid and lysolipid are combined in a container, e.g., a round bottom flask, using a pipette. The lipids are dried in the container, e.g., by blowing a thin stream of nitrogen gas into the container. The container is slowly rotated during the drying process to form a thin film on the container's surface. The thin film may be further dried by placing the container in a vacuum. The lipids are then re-liquified by adding the active agent in liquid form.

Next, the container is placed in a water bath at a temperature approximately equal to the transition temperature of the primary phospholipid. At this point, the container contains multilamellar vesicles with unecapsulated active agent. Sonication may be applied to the container and its contents to disrupt the lipid material, causing the multilamellar vesicles to open and resist the formation of unilamellar vesicles. Following the sonication, the multilamellar vesicles close around the active agent, encapsulating it within the aqueous core of the particle. In a preferred embodiment, the sonication provides ultrasound waves at approximately 20 kHz using an acoustic horn; the timing, magnitude, and duration of the ultrasonic energy are controlled. Active agent that remains unencapsulated should be removed, e.g., using a desalting column. Desalting columns are preferred because the particles may be sensitive to spin columns and centrifugation. Following the separation of unecapsulated active agent, the particles are ready for use.

### EXAMPLES

### Example 1

The experiment tested the percent release of encapsulated carboxyfluorescein from three different types of nanoparticles. The first type of nanoparticle included a DPPC bilayer with no lysolipid; the DPPC had a chain length of 16. The second type of nanoparticle included a DPPC and MMPC bilayer with a molar ratio of 95:5 DPPC to MMPC. The DPPC had a chain length of 16 and the MMPC had a chain length of 14, giving the bilayer a chain length difference of 2. The third type of nanoparticle included a DPPC and MPPC bilayer with a molar ratio of 95:5 DPPC to MPPC. The DPPC and the MPPC had identical chain lengths of 16 each.

The amount of liquid carboxyfluorescein released from the particles was measured using a fluorometer. The nanoparticles were injected in a thin (2 mm) closed transparent membrane containing 2.5 ml of nanoparticles mixed with a buffered solution. Control samples were reserved prior to ultrasonic visualization and insonation for measurement purposes. The closed membrane was immersed in room temperature water at a distance of 1.5 cm from the ultrasound probe and was slowly rotated. During the rotation, the ultrasonic energy was delivered to the nanoparticles within the closed membrane. Samples of the contents of the closed membrane were immediately extracted after the delivery of ultrasonic energy and were analyzed using a fluorometer.

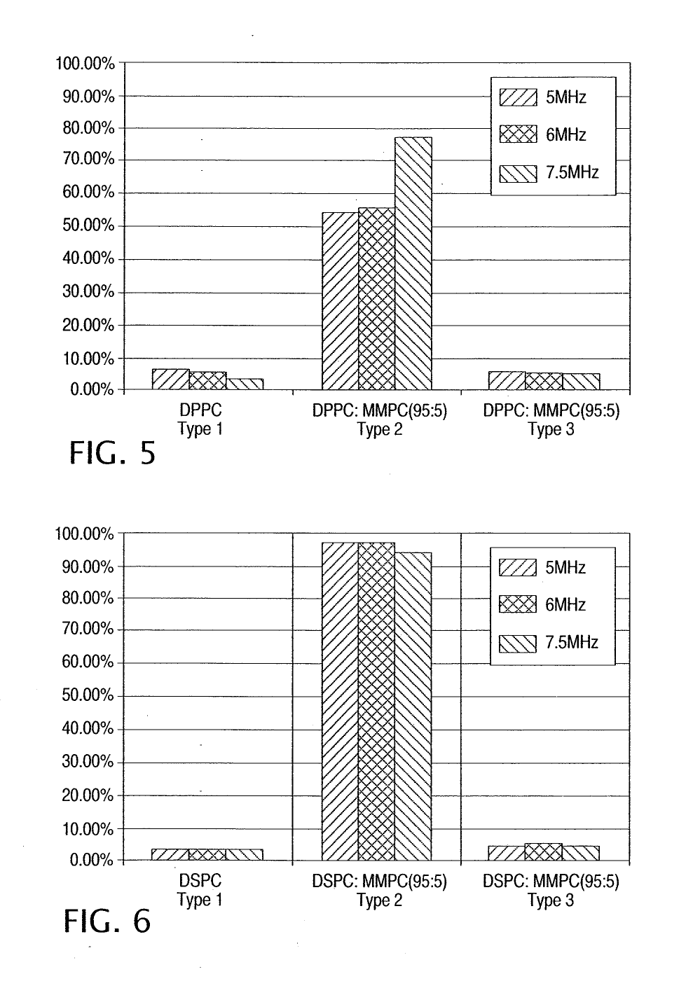

FIG. 5 provides a chart that shows percent release of encapsulated carboxyfluorescein from the three types of 100 nm nanoparticles using a fixed level of ultrasonic energy. The data reflect the average of three trials. The three types of nanoparticles were insonated with 3.5 MPa of pulsed ultrasonic energy for a period of 90 seconds, at frequencies of 5, 6, and 7.5 MHz. The data showed considerably higher percentages of release for the second type of nanoparticle compared to the first and the third.

This is most likely due to the incorporation of a lysolipid with a different chain length than the primary phospholipid.

### Example 2

The experiment tested the percent release of encapsulated carboxyfluorescein from three different types of nanoparticles. The first type of nanoparticle included a DSPC bilayer with no lysolipid; the DSPC had a chain length of 18. The second type of nanoparticle included a DSPC and MMPC bilayer with a molar ratio of 95:5 DPPC to MMPC. The DSPC had a chain length of 18 and the MMPC had a chain length of 14, giving the bilayer a chain length difference of 4. The third type of nanoparticle included a DPPC (16:0) and MPPC (16:0) bilayer with a molar ratio of 95:5 DPPC to MPPC. The DPPC and the MPPC had identical chain lengths of 16 carbon atoms each.

The amount of liquid carboxyfluorescein released from the particles was measured using a fluorometer. The nanoparticles were injected in a thin (2 mm) closed transparent membrane containing 2.5 ml of nanoparticles mixed with a buffered solution. Control samples were reserved prior to ultrasonic visualization and insonation for measurement purposes. The closed membrane was immersed in room temperature water at a distance of 1.5 cm from the ultrasound probe and was slowly rotated. During the rotation, the ultrasonic energy was delivered to the nanoparticles within the closed membrane. Samples of the contents of the closed membrane were immediately extracted after the delivery of ultrasonic energy and were analyzed using a fluorometer.

FIG. 6 provides a chart that shows percent release of encapsulated carboxyfluorescein from the three types of 100 nm nanoparticles using a fixed level of ultrasonic energy. The data reflect the average of three trials. The three types of nanoparticles were insonated with 3.5 MPa of pulsed ultrasonic energy for a period of less than one second, at frequencies of 5, 6, and 7.5 MHz. The data showed considerably higher percentages of release for the second nanoparticle compared to the first and the third. This is most likely due to the incorporation of a lysolipid with a different chain length than the primary phospholipid.

### Example 3

The following protocol was used to prepare a particle encapsulating carboxyfluorescein as the active agent:
1. Dissolve a primary phospholipid and a lysolipid in a 10 mg/mL solution of chloroform, which assists in preventing the formation of lipid spheres.
2. Calculate the required volume of liquid required to form a lipid bilayer based on molar percentages. For example:
Total concentration of the (DPPC:MMPC) lipid in a 95:5: 10 umoL
95/100*10=9.5 umoL
5/100*10=0.5 umoL
DPPC volume is: (9.5E-6 moL)*(734.05 g/moL)*(10 L/g)=697 uL
MMPC volume is: (0.5E-6 moL)*(467.58 g/moL)*(10 L/g)=23.3 uL
3. Pipette the calculated volumes of the primary phospholipid and lysolipid in a container, e.g., a round bottom flask.
4. Blow a thin stream of N₂ gas into the flask for approximately 2 seconds.
5. Seal the flask with parafilm and store in a freezer at approximately −20 degrees Celsius.
6. Dry the lipid solution by blowing a thin stream of N₂ gas into the flask and slowly rotating the flask to spread the film over a large area.
7. Place the flask in a vacuum for about an hour so that the lipid film completely dries out the traces of chloroform.
8. After about an hour, hydrate the lipid film with 1 mL of 100 mM carboxyfluorescein, so the final concentration of lipid is 10 umoL/mL
9. Place the flask in a water bath set at around the transition temperature of the primary phospholipid for about 15 minutes. Following this step, the flask will contain a solution of multilamellar vesicles (MLVs), having an average size of 100-200 nm, with unencapsulated carboxyfluorescein.
10. Sonciate the solution using a probe sonicator for approximately five minutes in a pulsing mode. The process of sonication causes the MLVs to open and resist the formation of unilamellar vesicles. The opening of the MLVs allows the carboxyfluorescein to enter the aqueous core of the particles.
11. Stop the sonication process, at which point the MLVs close and encapsulate the carboxyfluorescein. Some carboxyfluorescein may remain unencapsulated.
12. Use a PD-10 desalting column, which is a G-25 premade column, to remove the unencapsulated carboxyfluorescein. The carboxyfluorescein particles are susceptible to breaking in a spin column or during centrifugation; hence it is better to use a desalting premade column.
13. Create 2.5 mL of sample by combining 50 uL of lipid with 2450 uL of Hepes buffer.
14. Create 2.5 mL of detergent sample by combining 50 uL of lipid with 2400 uL buffer and 50 uL 20% Triton X-100.
15. Load the samples into an opticell and hit the opticells with ultrasound.

Whereas particular embodiments of this invention have been described above for purposes of illustration, it will be evident to those skilled in the art that numerous variations of the details of the present invention may be made without departing from the invention as defined in the appended claims.

What is claimed is:

1. A composition for delivering at least one active agent comprising at least one particle having an aqueous core and a lipid bilayer, wherein the lipid bilayer encapsulates the at least one active agent within the aqueous core.

2. The composition of claim 1, wherein the lipid bilayer comprises a primary phospholipid and a lysolipid.

3. The composition of claim 2, wherein the primary phospholipid has an acyl chain length, the lysolipid has an acyl chain length, and the acyl chain length of the primary phospholipid differs from the acyl chain length of the lysolipid.

4. The composition of claim 3, wherein the acyl chain length of the primary phospholipid ranges from about 6 to about 20.

5. The composition of claim 3, wherein the acyl chain length of the primary phospholipid is about 18.

6. The composition of claim 3, wherein the acyl chain length of the lysolipid ranges from about 6 to about 24.

7. The composition of claim 3, wherein the acyl chain length of the lysolipid is about 14.

8. The composition of claim 2, wherein the primary phospholipid is selected from the group consisting of DPPC, DSPC, and combinations thereof.

9. The composition of claim 2, wherein the lysolipid is selected from the group consisting of MMPC, MPPC, MOPC, MLPC, MSPC, and combinations thereof.

10. The composition of claim 2, wherein the molar ratio of primary phospholipid to lysolipid ranges from about 80:20 to 95:5.

11. The composition of claim 2, wherein the primary phospholipid has a transition temperature greater than about 50 degrees Celsius.

12. The composition of claim 2, wherein the primary phospholipid has a transition temperature ranging from about 50 to about 55.1 degrees Celsius.

13. The composition of claim 2, wherein the lipid bilayer further comprises cholesterol.

14. The composition of claim 1, wherein the at least one particle is selected from the group consisting of nanoparticles and liposomes.

15. The composition of claim 1, wherein the at least one particle has a width ranging from about 30 nm to about 5000 nm.

16. The composition of claim 1, wherein the at least one particle has a width ranging from about 100 nm to 200 nm.

17. The composition of claim 1, wherein the at least one particle is tracked using ultrasound.

18. The composition of claim 1, wherein the at least one particle is directed to a treatment site using a ligand.

19. The composition of claim 18, wherein the ligand comprises at least one antibody that attaches to an antigen.

20. The composition of claim 1, wherein the lipid bilayer releases the active agent when ultrasound reaches a release pressure.

21. The composition of claim 20, wherein the release pressure ranges from about 1½ MPa to about 5 MPa.

22. The composition of claim 20, wherein the release pressure is about 3 MPa.

23. The composition of claim 20, wherein the release pressure is maintained for a duration ranging from about 100 milliseconds to 900 milliseconds.

24. The composition of claim 20, wherein the frequency at the release pressure ranges from about 2 to 20 MHz.

25. The composition of claim 20, wherein the frequency at the release pressure is about 7.5 MHz.
